(19) United States
(12) Patent Application Publication
(10) Pub. No.: US 2001/0028991 A1
(43) Pub. Date: Oct. 11, 2001

(54) Exposure pattern data generation apparatus associated with standard cell library and charged beam exposure

(75) Inventors: Ryoichi Inanami, Kanagawa-ken (JP); Shunko Magoshi, Kanagawa-ken (JP)

Correspondence Address: Finnegan, Henderson, Farabow,, Garrett & Dunner, L.L.P., 1300 I Street, N.W., Washington, DC, 20005-3315, US

(73) Assignee: Kabushiki Kaisha Toshiba

(21) Appl. No.: 09/817,270

(22) Filed: Mar. 27, 2001

(30) Foreign Application Priority Data
Mar. 28, 2000 (JP) P2000-87930

(51) Int. Cl. G03C 5/00; A61N 5/00; G21G 5/00

(52) U.S. Cl. 430/296; 430/942; 250/492.2

(57) ABSTRACT

Logic synthesis is conducted for CP apertures 44 using standard cells corresponding to shaping holes 4 used in logic design of a system and placed at first placement positions on the respective CP apertures 44. A CP aperture 44 used for exposure is selected from among the CP apertures for which logic synthesis has been conducted. Second placement positions of the standard cells on a substrate which standard cells correspond to the shaping holes 4 provided on the selected CP aperture 44 and wiring routes the standard cells are calculated.

### CROSS REFERENCE TO RELATED APPLICATIONS

The subject application is related to subject matter disclosed in the Japanese Patent Application 2000-087930 filed in Mar. 28, 2000 in Japan, to which the subject application claims priority under the Paris Convention and which is incorporation by reference herein.

### BACKGROUND OF THE INVENTION

1. Field of the Invention

The present invention relates to a method of generating the fine pattern of a semiconductor device or a photomask using charged electron beams or charged ion beams and particularly relates to charged beam exposure of a character projection (CP) type.

2. Description of the Related Art

With an electron beam exposure technique, it is possible to process a fine pattern in sub-micrometers or less which cannot be manufactured by photolithography. The electron beam exposure technique is, therefore, essential to a semiconductor processing technique required to manufacture a more miniaturized, higher integrated, more complicated semiconductor. Invariable shaped beam (VSB) exposure which is a typical electron beam exposure method, a mask is not necessary regardless of the shape of a pattern for exposure. However, since the pattern is divided into many fine rectangular shots and exposure is repeatedly conducted, time required for exposure disadvantageously becomes long and desired throughput cannot be disadvantageously obtained.

To improve throughput, a character projection (or CP) exposure technique for allowing shots to be irradiated on a pattern of a certain degree of magnitude collectively, is developed. According to this technique, as shown in FIG. 1, an electron beam 41 is shaped rectangularly, a desired character is selected from a plurality of character-shaped beam shaping holes 49 formed on a CP aperture 50, the electron beam 41 is shaped into a desired character form and the demagnified electron beam 41 thus shaped is applied onto a desired portion of a substrate 37.

FIG. 1 shows that four types of characters are arranged on the CP aperture 50 and one of the four characters is selected. In case of the selected character, if VSB exposure is conducted, the character is divided into five very small rectangles and exposure is repeated five times in succession. If CP exposure is conducted, it suffices to conduct exposure once, so that the number of electron beam shots can be reduced to ⅕. Further, as for a pattern (or a character which is not arranged on the CP aperture) which cannot be subjected to exposure, VSB exposure is performed as conventional, so that a VSB shaping hole is also provided on the CP aperture 50.

The upper limit of the number of characters which can be selected during exposure is the number of characters which can be arranged in the deflected region of a character select deflector 43. Since each character is processed on the aperture to have a magnitude several times to several tens of times as large as the magnitude of the character exposed on a sample. For that reason, with an existing exposure system, only about a few to a hundred character scan be used for CP exposure. Conventionally, therefore, CP exposure is conducted to a pattern such as the pattern of a memory cell which has a large number of times of conducting exposure repeatedly; however, CP exposure cannot be conducted to the other patterns and only VSB exposure, though exposure time is long for VSB exposure, is applicable thereto.

Furthermore, in case of a logic product such as an application specific IC (or ASIC) and a system LSI, far more types of patterns than those of memories are used. Even if CP exposure is conducted to such many types of patterns and patterns are arranged on a CP aperture by the upper limit number of characters which can be subjected to CP exposure, the number of electron beam shots for each logic product varies depending on which pattern is made into a character. Some logic products cannot obtain the effect of improving throughput even if conducting CP exposure.

The layout of a logic product such as an ASIC (or pattern generation) is conducted according to a flow shown in FIG. 2.

First, in a step S31, the system specification of a product is decided. Next, in a step S32, a design is described in the form of a logic expression. In a step S33, using a logic synthesis system, the electronic circuit of a gate level which consists of logical function elements is generated.

In the logic synthesis in the step S33, the system described in the logic expression is converted into the connection of cell patterns (or net list) included in a conventional standard cell library 51. The cell patterns at this moment are selected appropriately based on signal transmission timing calculated from the function, resistance, capacity and the like of cells. FIG. 3 shows a net list. Standard cells 53 to 58 are differentiated from one another by cell names of AN2, E0 and FA1. The standard cells 53 to 58 are connected by wirings and function as a so-called integrated circuit as a whole. Next, in a step S34 shown in FIG. 2, logic simulation and timing analysis are performed by logic optimization and the circuit is corrected if there is an illegal part.

Then, in a step S35, using an automatic placement and routing tool, actual layout patterns are generated. In the step S35 of the automatic placement and routing (P&R), standard cells corresponding to the respective cell names of the net list are arranged, by referring to the conventional standard cell library 52. FIGS. 4A to 4C show the layout of the gate layer of the standard cells. In the following description, it is assumed that a gate layer is exposed with an electron beam. This is intended to avoid the repetition of description and to clarify the description. FIG. 4A shows the layout of an AND (AN2) circuit, FIG. 4B shows the layout of a D flip-flop (F/F) (FD1Q) circuit, FIG. 4C shows the layout of an inverter (IV), which layouts are stored in the library 52. In the step S35 of automatic placement and routing, the layouts of the standard cells as shown in FIGS. 4A to 4C are arranged in a substrate or a region 65 assumed as a photomask as shown in FIG. 5A so as to realize a logic circuit. Finally, routing is performed among the respective standard cells automatically, thereby completing a conventional layout.

To perform exposure based on this layout, it is necessary to convert conventional pattern data 70 on layouts into electron beam exposure data 75 as shown in FIG. 6. To do so, first, a character subjected to CP exposure is extracted. Conventionally, by observing the layout shown in FIG. 5A, it is discovered that there are same characters 61 to 64 and the character shown in FIG. 5B is selected as a character subjected to CP exposure. Information on the selected character is converted into CP exposure data 74, while unselected pattern data is converted into VSB exposure data 73.

If pattern data 70 is generated for each semiconductor device having integrated circuits, then it is necessary to execute a step 71 of extracting CP exposure characters for each integrated circuit and a step 72 of dividing the pattern data into the VSB exposure data 73 and the CP exposure data 74, which steps requires lots of time and labor.

### SUMMARY OF THE INVENTION

The present Invention has been made in view of the above-stated circumstances, It is, therefore, an object of the present invention to provide a charged beam exposure capable of conducting CP exposure to an integrated circuit having a few repetitive pattern such as a logic product, obtaining the effect of the improvement of throughput by conducting CP exposure and easily generating charged beam exposure data.

It is also an object of the present invention to provide an exposure pattern data generation method capable of conducting CP exposure even to an integrated circuit having a few repetitive pattern such as a logic product, obtaining the effect of the improvement of throughput by conducting CP exposure and easily generating charged beam exposure data and to provide an exposure pattern data generation apparatus for realizing this method.

To obtain the above objects, the first feature of the present invention lies in a charged beam exposure comprising:

a beam generation source generating a charged beam;

a CP aperture having shaping holes of the charged beam having shapes of standard cells used for design of logic device;

standard cell library recording means recording first placement positions of the shaping holes on the CP aperture;

pattern data recording means for recording second placement positions of the standard cells on a substrate, the second placement positions associated with the first placement positions;

a character select deflector irradiating the charged beam onto the shaping holes at the first placement positions; and

an objective deflector irradiating the charged beam onto the second placement positions on the substrate.

Here, “standard cell” means a pattern defined in the cell library. “Charged beam” means an electron beam or an ion beam.

According to the first feature, In case of particularly manufacturing a logic device such as an application specific IC (ASIC) or a system LSI, the shapes of patterns (or standard cells) defined in cell libraries used in a designed phase are manufactured on the aperture as electron beam shaping holes and the patterns are made into characters subjected to CP exposure, whereby electron beam exposure having higher throughput than conventional throughput is realized. Further, by making the standard cells into characters subjected to CP exposure, the number of electron beam shots can be sufficiently reduced and throughput can be, therefore, improved.

Standard cells allotted to the characters subjected to CP exposure do not have great change until the generation of logic products is changed. The CP aperture manufactured by placing the standard cells thereon can be used in common among a plurality of logic products. Due to this, if the logic products are designed using the same standard cell libraries, the same CP aperture can be always used for the logic products. Due to this, it is possible to start electron beam exposure right after generating layout data on a designed pattern. To deal with a case where the tendency of standard cells to be used greatly differs among the logic products, a method of manufacturing a plurality of CP apertures and replacing a CP aperture with another when electron beam exposure is conducted for the respective logic products or selecting a CP aperture in a different deflected region can be adopted. It is, therefore, possible to deal with more logic products.

The second feature of the present invention lies in an exposure pattern data generation apparatus comprising:

CP aperture decision means for conducting logic synthesis for CP apertures using standard cells corresponding to shaping holes placed on first placement positions on the respective CP apertures, and for selecting the CP aperture used for exposure; and

placement and routing means for calculating second placement positions of the standard cells on a substrate, the standard cells corresponding to the shaping holes provided on the selected CP aperture.

According to the second feature of the present invention, in case of particularly manufacturing a logic device such as an application specific IC (ASIC) or a system LSI, the shapes of patterns (or standard cells) defined in cell libraries used in a designed phase are manufactured on the aperture as electron beam shaping holes and the patterns are made into characters subjected to CP exposure, whereby electron beam exposure having higher throughput than conventional throughput is realized. Further, by making the standard cells into characters subjected to CP exposure, the number of electron beam shots can be sufficiently reduced and throughput can be, therefore, improved.

The third feature of the present invention lies in an exposure pattern data generation method comprising:

conducting logic synthesis for CP apertures using standard cells corresponding to shaping holes placed at first placement positions on the respective CP apertures;

selecting a CP aperture used for exposure from the CP apertures; and

calculating second placement positions of the standard cells on a substrate, the standard cells corresponding to the shaping holes provided on the selected CP aperture.

According to the third feature of the present invention, the characters subjected to CP exposure are stored in libraries as standard cells and patterns are selected from these characters in a designed phase, thereby making it possible to facilitate design and to shorten time required for data conversion to conduct electron beam exposure. Further, It is possible to check in a designed phase that pattern data can be generated by taking account of electron beam exposure in the phase of designing a logic product such as an ASIC and highest throughput can be obtained by using an appropriate aperture. Besides, if it is found that a new CP aperture needs to be manufactured, the manufacture of such a new CP aperture can be started when standard cells to be used are determined, so that a development period can be shortened even if manufacturing the CP aperture.

Other and further objects and features of the present invention will become obvious upon an understanding of the illustrative embodiments about to be described in connection with the accompanying drawings or will be indicated in the appended claims, and various advantages not referred to herein will occur to one skilled in the art upon employing of the invention in practice.

### BRIEF DESCRIPTION OF THE DRAWINGS

FIG. 1 is a conceptual view of a conventional CP type electron beam exposure;

FIG. 2 is a flow chart showing a layout method for a conventional semiconductor device;

FIG. 3 is a logic circuit diagram (net list) generated by the logic synthesis of the conventional semiconductor device layout method;

FIGS. 4A to 4C show the layouts of standard cells;

FIG. 5A shows a layout corresponding to pattern data generated by the placement and routing of the conventional layout method;

FIG. 5B shows a conventional character pattern;

FIG. 6 shows how to convert layout data into electron beam exposure data if executing the conventional layout method;

FIG. 7 is a conceptual view of a CP type exposure beam exposure according to one embodiment of the present invention;

FIG. 8 is a conceptual view of an exposure pattern data generation apparatus according to the embodiment of the present invention;

FIG. 9 is a flow chart showing conceptually the exposure pattern data generation method according to the embodiment of the present invention;

FIG. 10 is a hierarchically structural view of the data structure of an ASIC product generated using standard cell libraries storing CP information;

FIG. 11 is a data structural view of the standard cell library used for the CP type electron beam exposure method;

FIG. 12 is a block diagram of a CP aperture according to the embodiment of the present invention;

FIG. 13 is a block diagram of a CP aperture block according to the embodiment of the present invention;

FIG. 14 is a flow chart showing an exposure pattern data generation method according to one embodiment of the present invention in detail;

FIGS. 15A and 15B show a list of standard cells used in a semiconductor device according to Example 1 of generating exposure pattern data;

FIG. 16 is a graph showing the relationship between the number of characters subjected to CP exposure and the total number of electron beam shots;

FIG. 17 is a diagram of a logic circuit generated by the logic synthesis of the exposure pattern data generation method according to one embodiment of the present invention;

FIG. 18 shows a layout corresponding to pattern data generated by the placement and routing of the exposure pattern data generation method according to the embodiment of the present invention;

FIG. 19 shows how to convert layout pattern data into electron beam data; and

FIG. 20 is a table showing the effect of a reduction In the number of electron beam shots and the effect of improvement in the throughput of an exposure for a semiconductor device B according to Example 2 of generating exposure pattern data.

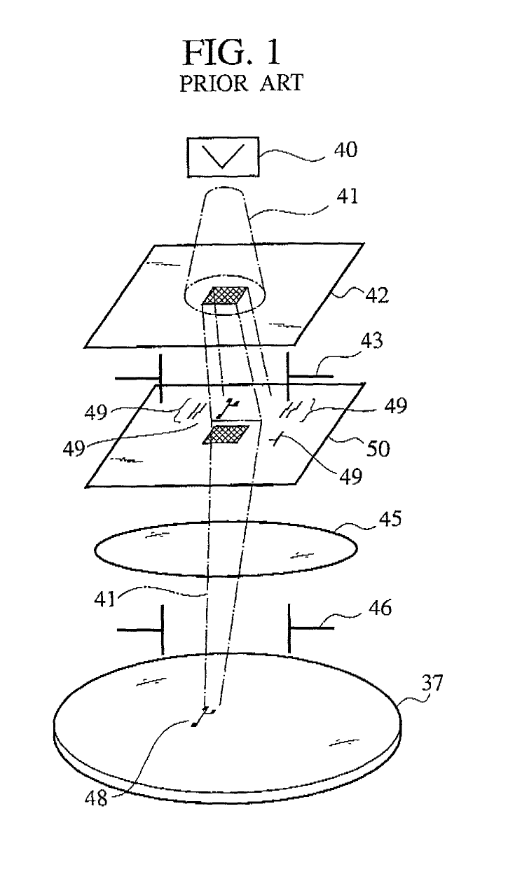

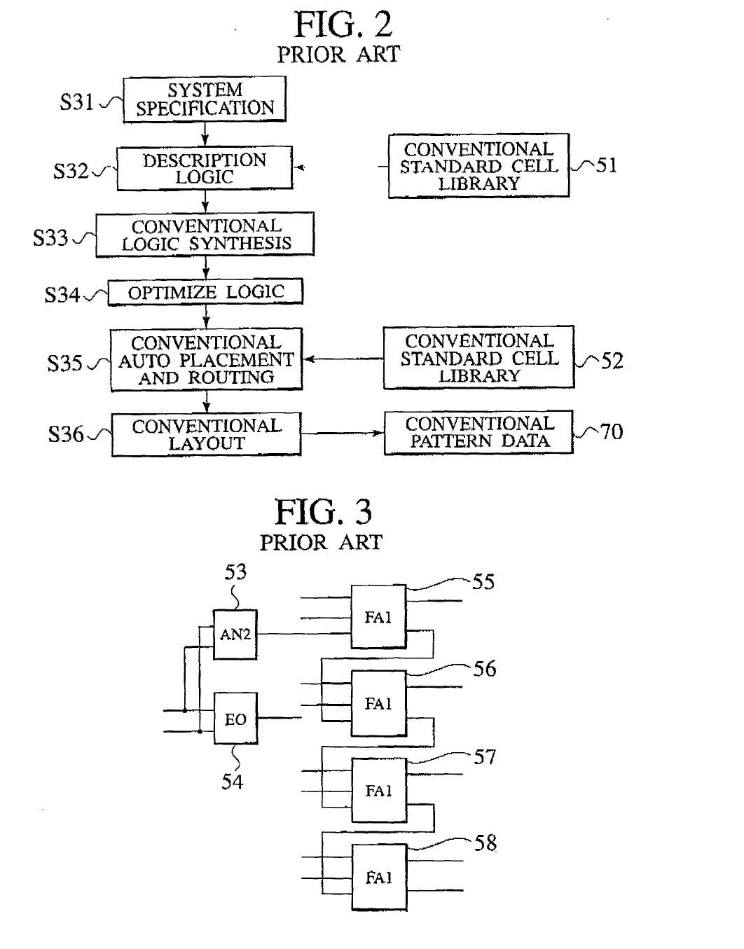

### DETAILED DESCRIPTION OF THE EMBODIMENTS

Various embodiments of the present invention will be described with reference to the accompanying drawings. It is to be noted that the same or similar reference numerals are applied to the same or similar parts and elements throughout the drawings, and the description of the same or similar parts and elements will be omitted or simplified.

FIG. 7 is a conceptual block diagram of a charged beam exposure according to the present invention. The charged beam exposure according to the present invention has an electron gun 40 generating an electron beam 41, the first shaping aperture 42 shaping an irradiation pattern of the electron beam 41 into a rectangle, a character select deflector 43 deflecting the rectangular electron beam 41 and irradiating the deflected rectangular electron beam 41 onto electron beam shaping holes 4 each of a standard cell shape, a CP aperture or CP aperture block 44 having the shaping holes 4 and a VSB opening 7, a demagnification lens 45 demagnifying the electron beam 41 shaped by the shaping holes 4, and an objective deflector 46 deflecting the electron beam 41 and irradiating the shaped electron beam 41 onto desired positions on a substrate or photomask 47.

Also, the charged beam exposure may have character select deflector control means 81 connected to the character select deflector 43, objective deflector control means 82 connected to the objective deflector 46, standard cell library recording means 102 connected to the control means 81 and recording circuit pattern placement information and CP information, and pattern data recording means 103 connected to the control means 81 and 82 and recording pattern data associated with the CP information.

The character select deflector control means 81 controls the deflector 43 and irradiates the shaping holes 4 of standard cells corresponding to the pattern data with the electron beam 41 based on the CP information recorded on the cell library recording means 102 and the pattern data associated with the CP information recorded on the pattern data recording means 103. The objective deflector control means 82 controls the deflector 46 and irradiates placement positions on the substrate or the like 47 corresponding to the pattern data with the electron beam 41 based on the placement positions of the standard cells of the pattern data recorded on the pattern data recording means 103.

FIG. 7 shows that the electron beam shaping holes 4 of four types of standard cell shapes are placed on the CP aperture 44 and that one of the electron beam shaping holes 4 is selected. If the character of this selected shaping hole 4 is subjected to VSB exposure, the character is divided into several tens of very small rectangles and exposure is conducted to the divided rectangles repeatedly in succession. In case of CP exposure, by contrast, it suffices to conduct exposure once for every character. As for a pattern which cannot be subjected to CP exposure (or which is not placed on the CP aperture), VSB exposure is conducted as conventional. For that reason, the VSB shaping hole 7 is also provided on the CP aperture 44. By irradiating the rectangular electron beam onto part of the shaping hole 7, a rectangular pattern of a desired magnitude can be irradiated onto the substrate or the like 47.

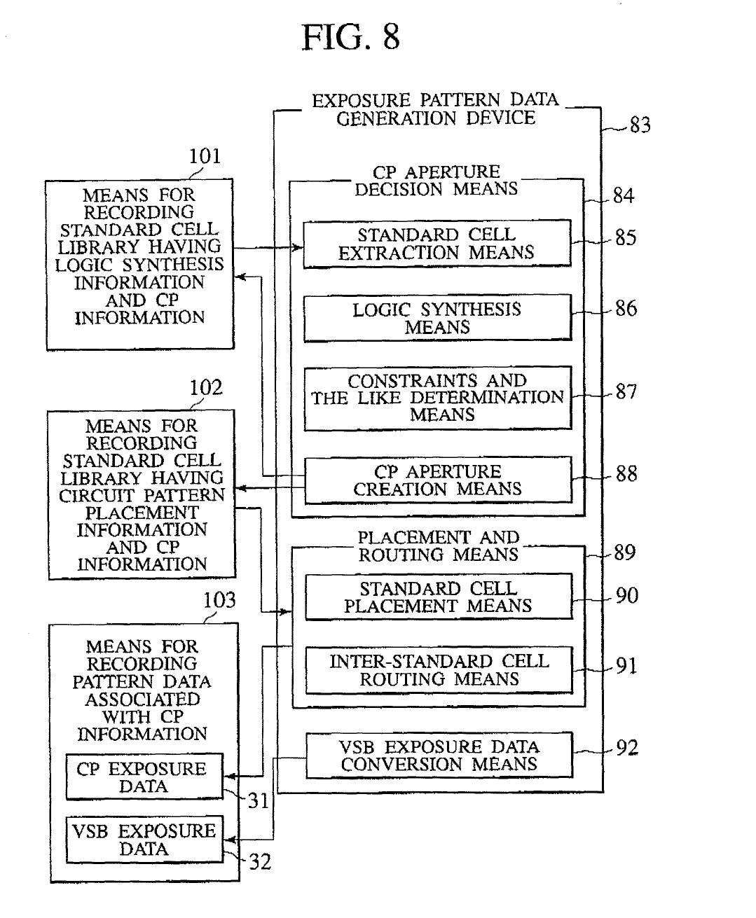

FIG. 8 is a conceptual block diagram of an exposure pattern data generation apparatus 83 according to the present invention. The exposure pattern data generation apparatus 83 according to the present invention has CP aperture decision means 84 for conducting logic synthesis using standard cells corresponding to the shaping holes 4 provided on the CP aperture 44, and for selecting a CP aperture 44 used for exposure in the course of the production of a semiconductor device or the production of a photomask used for the production of the semiconductor device, and placement and routing means 89 for calculating positions at which the standard cells corresponding to the shaping holes 4 provided on the selected CP aperture 44 are placed and the positions of wiring routes provided among the cells. It is preferable that the generation apparatus 83 also has VSB exposure data conversion means 92 for converting data into data which can be used in the exposure to conduct VSB exposure to a standard cell to which CP exposure cannot be conducted using the shaping holes 4.

The CP aperture decision means 84 has standard cell extraction means 85 for extracting the standard cells corresponding to the shaping holes 4 provided on a certain CP aperture 44, logic synthesis means 86 for conducting logic synthesis using the extracted standard cells corresponding to the certain CP aperture 44, constraints and the like determination means 87 for determining whether the system of a semiconductor device or the like, for which the logic synthesis corresponding to the certain CP aperture 44 has been performed, satisfies constraints including specification, the throughput of an exposure step and the number of exposure shots, and CP aperture creation means 88 for creating a new CP aperture 44 if the existing CP aperture 44 cannot satisfy the constraints.

The placement and routing means 89 has standard cell placement means 90 for calculating positions at which the standard cells corresponding to the shaping holes 4 provided on the selected CP aperture 44 are placed, and inter-standard cell routing means 91 for calculating the positions of wiring routes provided among the standard cells.

Further, the exposure pattern data generation apparatus 83 may have standard cell library recording means 101 having logic synthesis information and CP information, standard cell library recording means 102 having circuit placement information and CP information, and pattern data recording means 103 having pattern data associated with the CP information. Pattern data consisting of CP exposure data 31 used for CP exposure and VSB exposure data 32 used for VSB exposure are recorded on the recording means 103.

The recording means 101 is connected to the CP aperture decision means 84 and provides information for the logic synthesis of standard cells and the CP information to the decision means 84. The logic synthesis information involve the magnitudes, functions, performances, driving forces and the like of the standard cells. The CP information involve discrimination codes such as the names of the CP aperture 44 on which the shaping holes 4 each having a standard cell shape are formed, and the positions at which the shaping holes 4 are placed on this CP aperture 44. Further, the recording means 101 is connected to the CP aperture creation means 88. The creation means 88 provides to the recording means 101 the discrimination code of a newly created CP aperture 44, the names of standard cells corresponding to the shapes of shaping holes 4 formed on the newly created CP aperture 44 and the placement positions of the shaping holes 4.

The recording means 102 is connected to the placement and routing means 89 and provides circuit placement information and CP information to the placement and routing means 89. The circuit placement information involve the shapes, magnitudes of the outlines, input/output positions and the like of the standard cells. As in the case of the recording means 101, the recording means 102 is connected to the CP aperture creation means 88 and receives the same information as that the recording means 101 receives.

The recording means 103 is connected to the placement and routing means 89 and the VSB exposure data conversion means 92. In case of a gate layer or the like which does not include a wiring pattern, the discrimination codes of the standard cells to be placed and the placement positions are provided, as CP exposure data 31, from the placement and routing means 89 to the recording means 103. In case of a layer including a wiring pattern, the positions of wirings provided among the standard cells as well as the above-stated data are provided from the placement and routing means 89 to the recording means 103. Unlike the CP exposure data 31, the shapes of the cells instead of the discrimination codes of the standard cells to be placed are provided as the pattern data of the VSB exposure data 32.

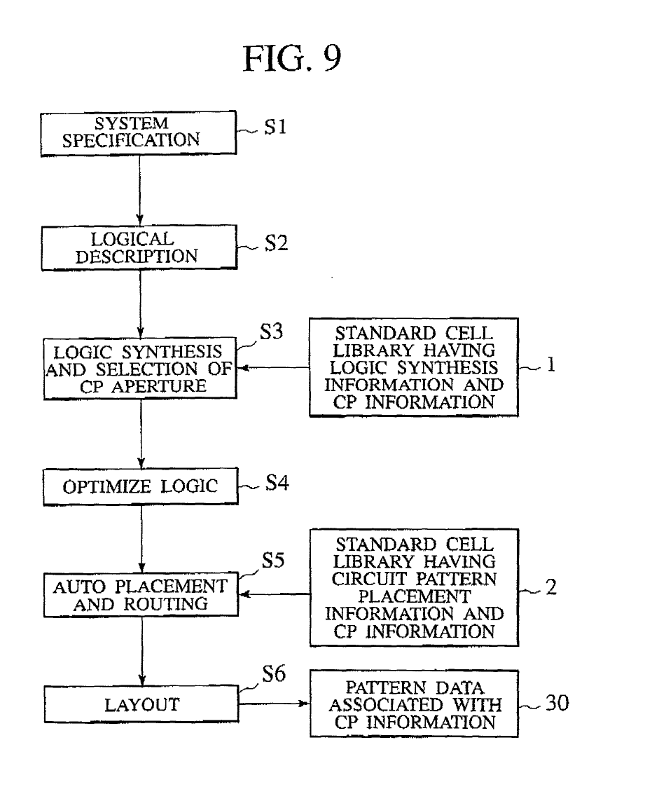

The designed pattern of a logic product represented by an ASIC which is one of semiconductor devices is created according to a flow as shown in FIG. 9. Here, the simulation, timing analysis and the like in the respective steps are omitted because they are not so relevant to the present invention. In a step S1, the system specification of a semiconductor device product is decided. In a step S2 of logical description, an electronic circuit is described in the form of a logic expression. In a step S3 of logic synthesis and CP aperture selection, a design description for a gate level or the like is generated using a logic synthesis system. In a step S3, the CP aperture decision means 84 shown in FIG. 8 converts the electronic circuit described in the logic expression into the connection of cell patterns (or net list) included in the standard cell library having the logic synthesis information and the CP information recorded on the recording means 101. At this time, an appropriate cell pattern is selected based on signal transmission timing calculated from the functions, resistances and capacities of the cells or the like.

Next, in a step S4 of logic optimization, logic simulation and timing analysis are conducted and the circuit is corrected if there is an Illegal part. Then, in a step S5 of automatic placement and routing, an actual layout pattern is generated using the placement and routing means 89 shown in FIG. 8. In the automatic placement and routing (P&R). the standard cells corresponding to the net list are placed and wirings among the standard cells are automatically provided by referring to the standard cell library 2 having the CP information recorded on the recording means 102. As a result, designed pattern data 30 associated with the CP information which is on the layout of a semiconductor device such as a logic product is completed. The completed data 30 is recorded and stored by the recording means 103 shown in FIG. 8.

Each standard cell has information for logic synthesis, information for circuit placement and CP information. The logic synthesis information is data derived from the library 1 shown in FIG. 9 such as, for example, the magnitude, function, performance and the like of the cell. The circuit placement information is data derived from the library 2 shown in FIG. 9 such as, for example, the concrete shape of the gate layer of a circuit, the positions of input and outputs connected to wirings and the like. As the CP information, which position this cell is placed on is described. Therefore, some cells are placed on a plurality of apertures and the others are not placed on any aperture and do not include the CP information.

The created designed layout pattern 30 gradually creates a cell hierarchical structure as the pattern becomes larger in size. Since a portion subjected to CP exposure on a gate layer or the like does not include wirings provided among the cells, all patterns subjected to CP exposure in the portion can be shot with every standard cell. For example, even if pattern data is of a GDSII STREAM type which is normally used, the data 30 can be used as it is because it suffices to get only the positions at which the standard cells subjected to CP exposure are placed.

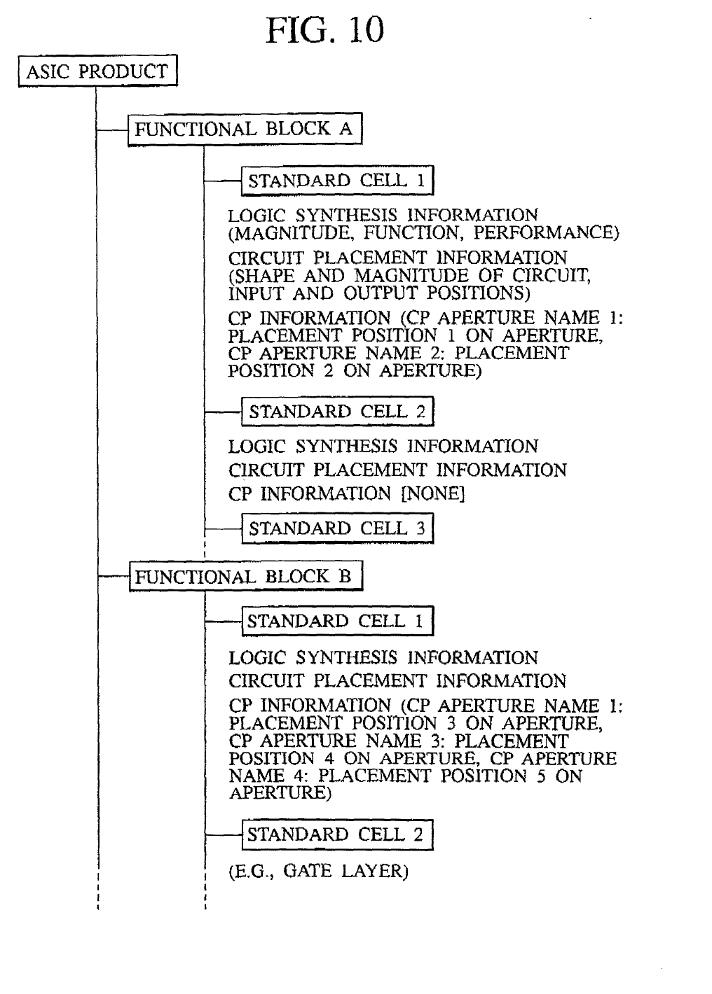

FIG. 10 shows the data structure of the designed pattern of an ASIC product using the standard cell libraries 1 and 2 having the CP information. Although the libraries 1 and 2 are described separately because they differ in data derived therefrom, a common recording region may be used and the two libraries 1 and 2 shown in FIG. 9 maybe generally called a standard cell library. According to the data structure shown in FIG. 10, each of a plurality of functional blocks constituting this ASIC product consists of many standard cells. As for those standard cells, if the same standard cell library has been referred to for logic synthesis, some standard cells may have CP information and the others may not have CP information, depending on a CP aperture assumed to be used for exposure as shown in FIG. 10.

For the purpose of simplifying designing pattern, simplifying the conversion of the created layout pattern data 30 into the exposure data within each electron beam exposure and thereby reducing conversion time, it is preferable that standard cell libraries 1 and 2 for electron beam exposure are newly created and that automatic P&R is conducted using the libraries 1 and 2 to thereby generate layout data 30. The electron beam exposure standard cell libraries 1 and 2 may indicate which of the standard cells in the libraries are made into characters subjected to CP exposure and the positions of such standard cells on the CP aperture. In other words, CP information may be added to the conventional libraries for every standard cell.

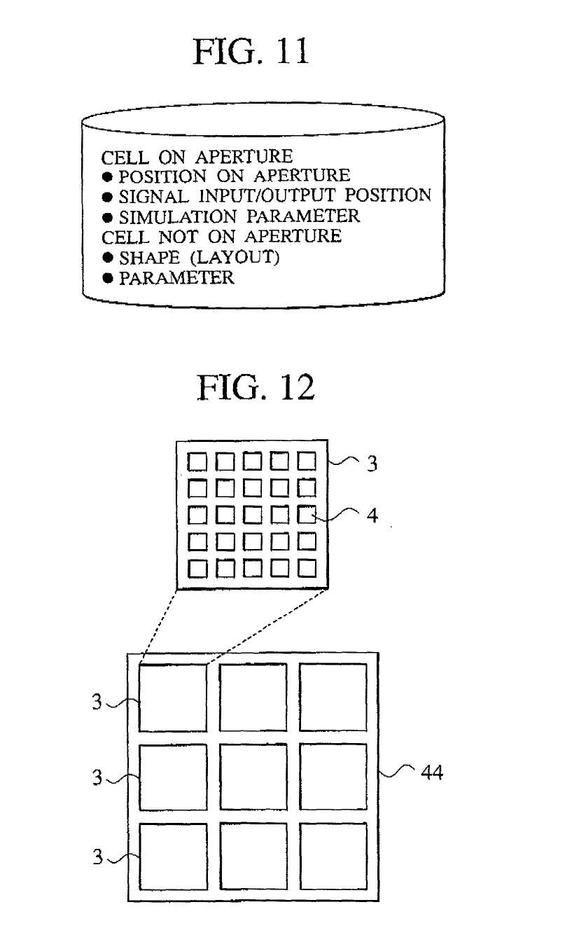

FIG. 11 shows the data structure of the electron beam exposure standard cell libraries 1 and 2. In this data structure, information as to whether or not a standard cell is placed on the aperture is stored for each standard cell. In addition, if the cell is placed on the aperture, information on the position of the cell on the aperture, information on signal input/output positions, parameter information indicating the function and performance of the cell used for the simulation of an integrated circuit are stored in the data structure. If the cell is not placed on the aperture, the detailed layout of the cell and the above-stated parameter information are stored, as well.

Each of the standard cell libraries 1 and 2 normally consists of several hundreds of standard cells . The design of an ordinary ASIC product is made by the automatic P&R of these standard cells. As for a large-scale product, hierarchical design for conducting automatic P&R for each functional block and then integrating the blocks is applied.

Accordingly, the pattern data 30 on the ASIC product designed by the above-stated method mainly consists of standard cells and layers in each cell are defined only within each standard cell. By contrast, the pattern data 30 on which wirings are provided by the automatic P&R to connect the respective standard cells is not defined in the standard cells and generated using information on the input/output positions of the cells.

That is, it is obvious that if a pattern, such as a gate layer pattern, which exists only in the individual standard cell is exposed by the CP type electron beam exposure method and each of the standard cells defined in the standard cell libraries 1 and 2 is set as a character subjected to CP exposure, then all of the pattern data 30 can be created by the CP exposure.

Next, description will be given to a method of designing the circuit pattern of a semiconductor device such as a logic product or the like and a method of generating the layout pattern data thereof using the standard cell libraries 1 and 2 referring to the CP aperture 44. Namely, as the pattern becomes larger in scale, the types of standard cells to be used and the frequency of using the standard cells increase and the hierarchical structures of the cells are created accordingly. All portions subjected to CP exposure serve as the units of the respective standard cells. Since it suffices to know the positions at which the standard cells subjected to CP exposure are placed, the libraries can be used as they are.

If different CP apertures 44 are selected for different products and CP exposure is conducted, information as to on which CP apertures 44 the respective standard cells are formed is provided, so that the following pattern design can be made.

The exposure shown in FIG. 7 may have a plurality of aperture blocks 3 respectively placed in regions which the character select deflector 43 can deflect like FIG. 12. In case of conducting electron beam exposure to a logic product which tends to employ different standard cells, it is possible to provide the exposure with a mechanism for mechanically moving the CP aperture 44 so that the standard cells formed in the different aperture blocks 3 can be selected. If a plurality of aperture blocks 3 on the CP aperture 44 or if a plurality of CP apertures corresponding to a plurality of products are provided, the following procedures are taken:

(1) Automatic P&R is conducted using the electron beam exposure standard cell library 2.

(2) It is checked on which CP aperture 44 and on which aperture block 3 a selected standard cell is formed as a character subjected to CP exposure, and a CP aperture 44 and an aperture block 3 are selected so that the number of shots of the electron beam 41 becomes a minimum.

(3) As for the standard cells formed on the selected CP aperture 44, the positions of the standard cells on the CP aperture 44 are outputted as pattern data 30, As for the other standard cells, which are subjected to VSB exposure, polygon data on the pattern is outputted as the pattern data 30.

Further, if there is only one type of a generalized CP aperture 44 or if a certain CP aperture 44 and a certain aperture block 3 are selected from among a plurality of CP apertures 44 to be used in advance for some reason, the following procedures are taken:

(1) Logic synthesis is conducted preferentially using the standard cells placed on the aperture 44.

(2) As for the standard cells of (1) above, the positions of the standard cells on the CP aperture 44 and on the aperture block 3 are outputted. As for the other standard cells, polygon data on the pattern is outputted for VSB exposure.

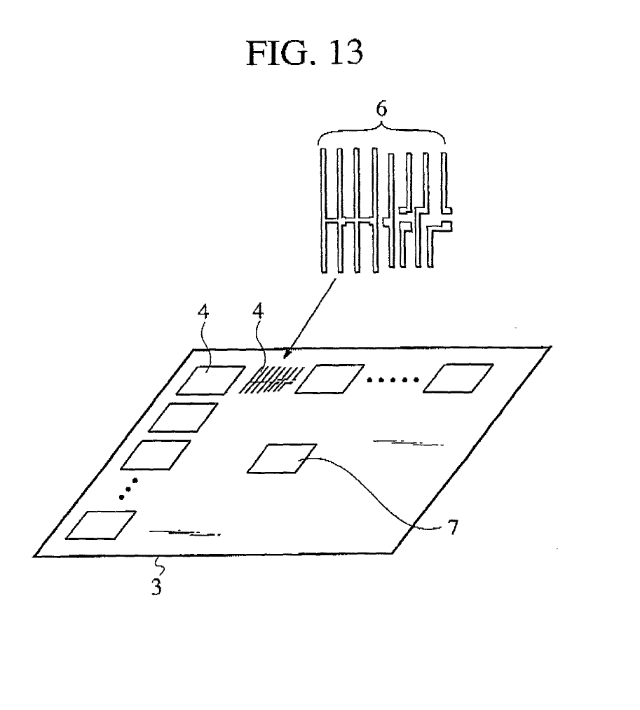

In the libraries, pattern shapes 6 are defined in all of the standard cells. Characters subjected to CP exposure are used as the units of the respective standard cells. As for each standard cell subjected to CP exposure, the pattern shape 6 is defined as the shaping hole 4 on the CP aperture block 3 as shown in FIG. 13. It suffices to know the positions at which the respective cells are placed on the chip of the semiconductor device. In addition, each CP aperture block has an opening 7 for VSB exposure.

As can be seen, the CP aperture block has charged beam shaping holes 4 each having the shape of a standard cell used for device design. It is more advantageous If the shaping holes 4 are in the shape of standard cells having higher frequency of use or standard cells having higher effects of reducing the number of shots for CP exposure than the number of shots for VSB exposure. Due to this, the standard cells within the cell libraries do not change according to products but standard cells are commonly used for a plurality of products. It is, therefore, unnecessary to create a mask for use in lithography for each product, which is cost effective, and it is also possible to start exposure right after obtaining designed pattern data. Further, even if the number of characters which can be placed on a CP aperture is limited and all standard cells cannot be made into characters subjected to CP exposure, it is possible to check which standard cells subjected to CP exposure allow effectively reducing the number of shots if exposure is executed using the same CP aperture for a plurality of products by examining the CP effectiveness of standard cells for at plurality of products, and to, therefore, manufacture a CP aperture which can be used among a plurality of logic products.

It is more advantageous if the aperture block 3 has a VSB opening. If so, both CP exposure and VSB exposure can be utilized for one semiconductor device. Particularly, if a standard cell which is used less frequently or a standard cell which has a low effect of reducing the number of shots is subjected to VSB exposure, it is possible to reduce the number of characters placed on a CP aperture without greatly deteriorating throughput.

Now, description will be given to a method of generating pattern data 30 suited for electron beam exposure using the electron beam exposure standard cell libraries 1 and 2 having CP information when CP type electron beam exposure is conducted using the CP aperture 44 having standard cell shapes. This generation method corresponds to the steps S3 to S5 shown in FIG. 9 and particularly relates to the logic synthesis method in the step S3 and the placement and routing method in the step S5.

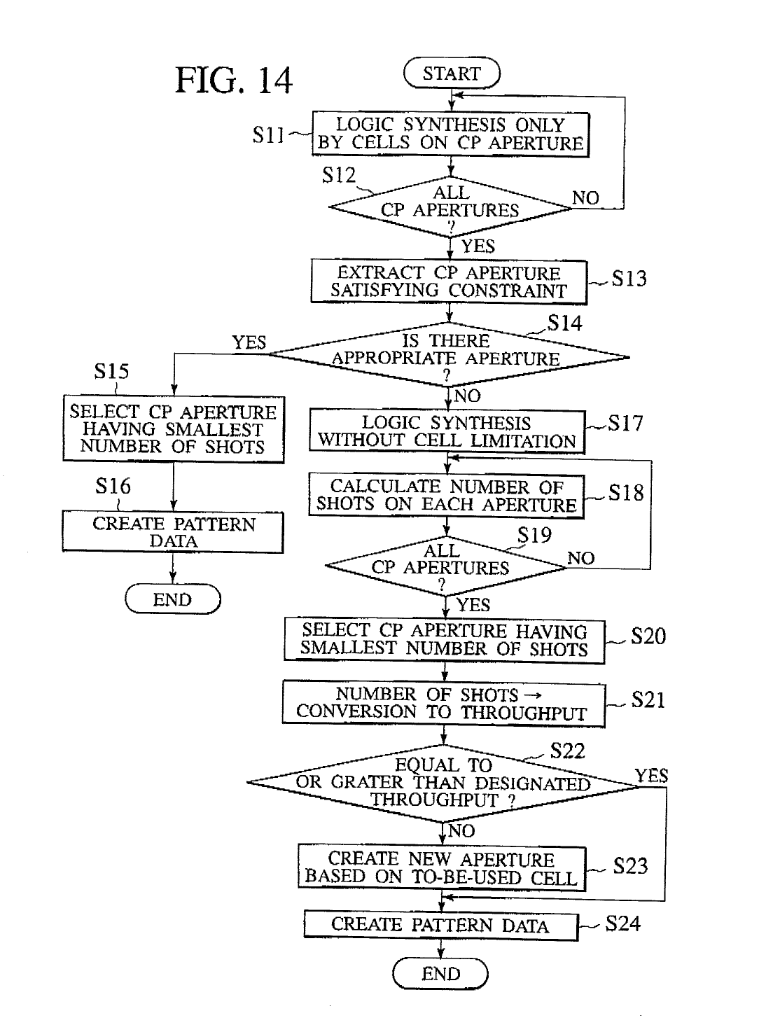

FIG. 14 is a flow chart for generating electron beam exposure pattern data. In the description of respective steps, an aperture 44 is equivalent for an aperture block corresponding to one deflected region. If there are a plurality of aperture blocks on the CP aperture 4, the aperture 44 indicates each aperture block 3.

First, in a step S11, assuming that exposure is performed using a certain aperture 44 and the logic synthesis of the electronic circuit shown in FIG. 9 is performed. In the logic synthesis, the standard cell extraction means 85 shown in FIG. 8 extracts only the cells subjected to CP exposure, i.e., only the standard cells placed on the CP aperture 44. Using the extracted standard cells, the logic synthesis means 86 conducts logic synthesis. At this moment, in the step S1 and the like shown in FIG. 9, the area of a synthesized electronic circuit, the operating frequency of the circuit and the like are designated as design constraints in advance.

In a step S12, a processing returns to the step S11 until the logic synthesis in the step S11 is conducted to all of the CP apertures.

In a step S13, the constraints and the like determination means 87 extracts CP apertures which satisfy the designated constraints from net lists synthesized in the steps S11 and S12. That is, the means 87 extracts CP aperture candidates which can be used.

In a step S14, the constraints and the like determination means 87 judges whether there are CP apertures satisfying the constraints if logic synthesis is conducted only to the cells placed on the CP apertures. If there are such CP aperture which satisfies the constraints, a step S15 follows. If not, a step S17 follows.

In the step S15, the constraints and the like determination means 87 selects a CP aperture 44 for which the number of electron beam shots become a minimum from the CP apertures 44 which can be used.

In a step S16, the placement and routing means 89 conducts P&R using the net list which has been synthesized for the CP aperture 44 selected in the step S15 and generates pattern data 30. Then, the processing flow is finished. Here, if electron beam exposure is conducted to the pattern data 30 generated in this flow using the selected CP aperture 44, all patterns can be formed by CP exposure.

On the other hand, if the step S17 follows, it means that the constraints and the like determination means 87 judges that patterns cannot be synthesized only standard cells arranged on the existing CP apertures 44. Due to this, the standard cell extraction means 85 eliminates the constraint of using only the cells on the CP apertures 44 and the logic synthesis means 86 conducts logic synthesis again.

In a step S18, the constraints and the like determination means 87 calculates the number of electron beam shots if exposure is conducted for the net list synthesized in the step S17 using each CP aperture 44. At this moment, the calculation is made by assuming that the cells placed on the CP aperture to be used are subjected to CP exposure and that the other cells are subjected to VSB exposure.

In a step S19, the calculation of the number of shots executed in the step S18 is made for all the CP apertures 44.

In a step S20, the constraints and the like determination means 87 selects the CP aperture 44 for which the number of shots calculated in the step S18 becomes a minimum.

In a step S21, the constraints and the like determination means 87 converts the number of shots for the CP aperture 44 selected in the step S20 into throughput. The throughput can be simply calculated according to Expression 1:

Throughput (number of wafers/h)=3600/(shot cycle×number of shots×number of chips×wait time) (1)

Here, time used in calculation is in units of seconds. The shot cycle is a cycle for repeating exposure, that is, the sum of actual electron beam irradiation time and beam deflection wait time. Further, the number of shots Is in the units of shots per chip since normal pattern design is made in the units of chips. Therefore, the number of chips is the number per wafer. The wait time is the sum of wait time of the other overhead, i.e., a sum per wafer of frame return time, main deflection wait time, mark detection and beam alignment time. The shot cycle and wait time are values determined according to process conditions and the electron beam exposure to be used. Therefore, when designing a pattern or generating pattern data, it is necessary to take into account of the following process and the exposure to be used. Alternatively, calculation may be made based on an ordinary process and ordinary exposure conditions, the calculated values may be converted into those for actual exposure and then throughput may be estimated.

In a step S22, the constraints and the like determination means 87 determines whether or not the throughput calculated in the step S21 is equal to or higher than desired throughput.

If the determination result in the step S22 snows that the throughput is lower than the desired throughput, the CP aperture creation means 88 extracts the standard cells having higher CP effectiveness to lower CP effectiveness included in the net list synthesized in the step S17 in this order and creates a new CP aperture in a step S23.

In a step S24, if it is determined in the step S22 that the desired throughput can be obtained, the placement and routing means 89 and the VSB exposure data conversion means 92 generate pattern data 30 using standard cells arranged on the CP apertures 44 selected in the step S20 or the CP aperture 44 newly created in the step S23. Then, the processing flow is finished.

A series of processings shown in the flow charts of FIGS. 9 and 14 are recorded as a program executed by a computer on a computer readable recording medium. Further, the data structures having data operated by the computer as shown in FIGS. 10 and 11 are recorded on the computer readable recording medium. The program is recorded on the exposure pattern data generation apparatus 83 shown in FIG. 8, while the data structures are recorded on the recording means 101 and 102. Here, the recording mediums involve, for example, mediums which can record programs such as a semiconductor memory, a magnetic disk, an optical disk and a magnetic tape. The data having the program and data structures is read by a computer system and the computer system conducts shape simulation according to the procedures described in the program. The computer system is provided with a floppy disk drive and a CD-ROM drive. By inserting a floppy disk or a CD-ROM from the corresponding drive inlet and conducting a predetermined read operation, the program stored in the recording medium can be installed into the computer system. Moreover, the data having the program and the data structures can be transferred between the computers through a transmission medium such as the Internet. Further, by connecting a predetermined drive to the computer system, it is possible to employ an ROM as a semiconductor memory used for, for example, a game pack or a cassette tape as a magnetic tape. In other words, the recording mediums involve mediums capable of recording programs such as a semiconductor memory, a magnetic disk, an optical disk and a magnetic tape.

Polygon data on the standard cells subjected to CP exposure can be omitted so as to prevent data size from becoming very large. As for the pattern data for which file size is reduced by omitting the polygon data, therefore, it is possible to download and upload the used design data using a network such as the Internet in a short time. Thus, it is possible to relatively easily order a product outside a company or to conduct process outside the company, which operations have been conventionally difficult.

This flow is used by the exposure pattern data generation apparatus 83. The CP aperture decision means 84 of the generation apparatus 83 executes steps S11 to S15 steps S17 to S23. Namely, the CP aperture decision means 84 decides on an aperture 44 used for CP exposure and standard cells subjected to the CP exposure using information on the standard cells which shapes are conformed to those of shaping holes 4 provided on the aperture 44, and conducts the logic synthesis of an electronic circuit. The placement and routing means 89 and the conversion means 92 execute steps S16 and S24. The placement and routing means 89 places and routes standard cells subjected to the CP exposure using information on the standard cells. The conversion means 92 converts the portions other than the standard cells subjected to CP exposure into VSB exposure data.

While generating pattern data 30 for exposure, it is possible to decide which CP aperture 44 among the CP apertures 44 is used for exposure or whether a new CP aperture 44 is manufactured.

That is, it is possible to generate pattern data 30 for which electron beam exposure is taken into consideration in the phase of designing a logic product such as an ASTC. It is also possible to check that highest throughput can be obtained by using an appropriate CP aperture 44 in the design phase. Further, if it is found that it is necessary to manufacture a new CP aperture 44, the CP aperture 44 can be manufactured at a time when standard cells to be used are defined. With this method, a development period can be advantageously reduced compared with a case where a light exposure mask cannot be manufactured until a final pattern layout is decided.

### Example 1 of Generating Exposure Pattern Data

The standard cell libraries 1 and 2 used in this example of generating exposure pattern data include about 400 standard cells. If all of the standard cells can be placed as characters on a CP aperture 44 or an aperture block 3, they can be subjected to CP exposure using the same standard cell libraries 1 and 2 for products designed by automatic P&R.

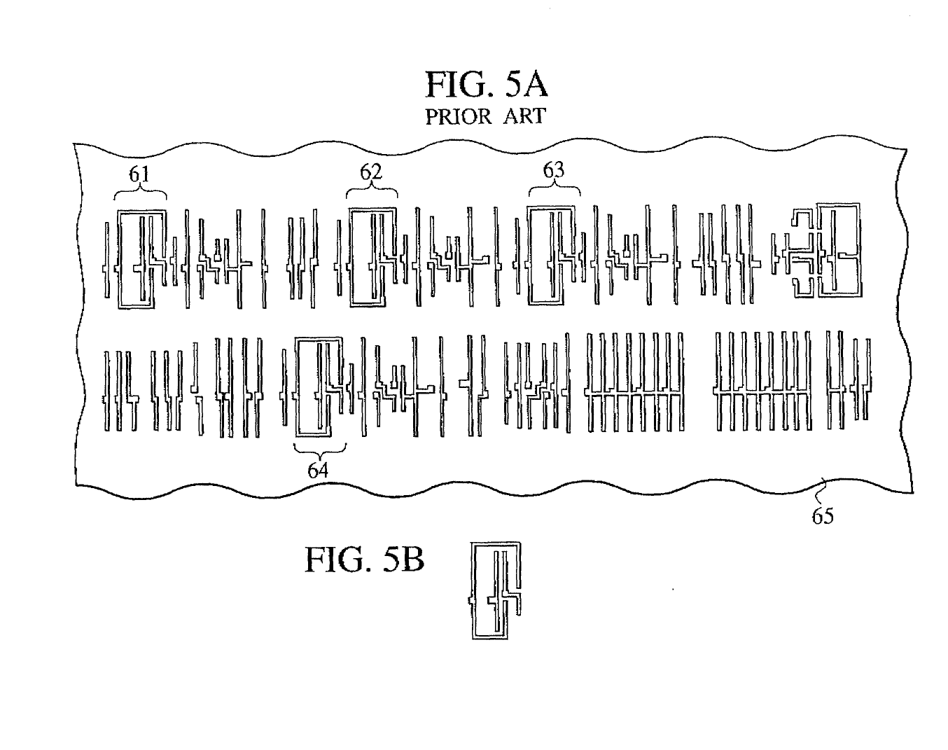

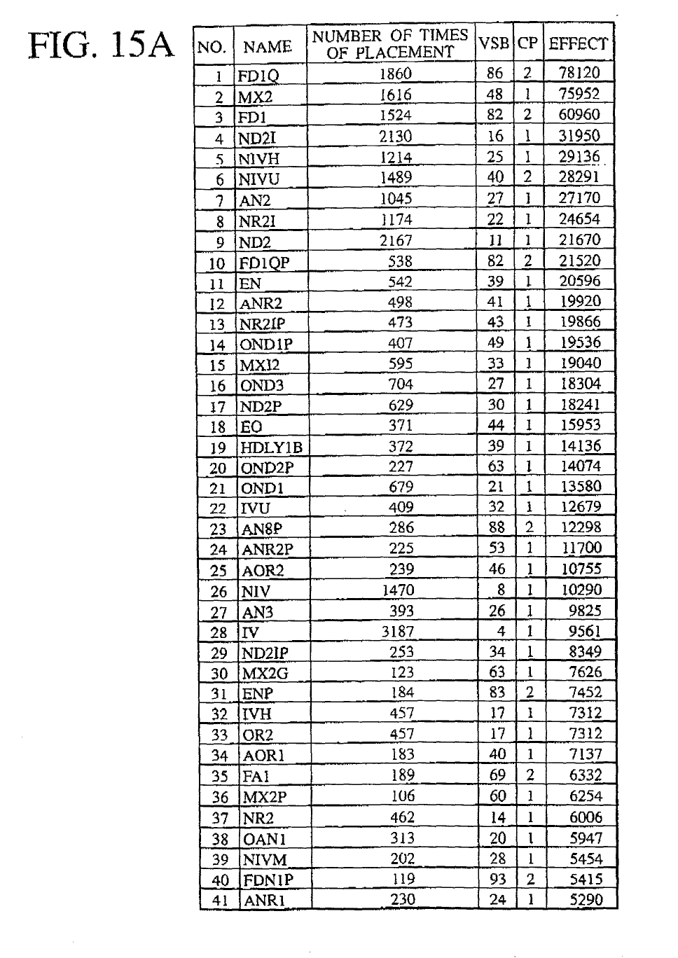

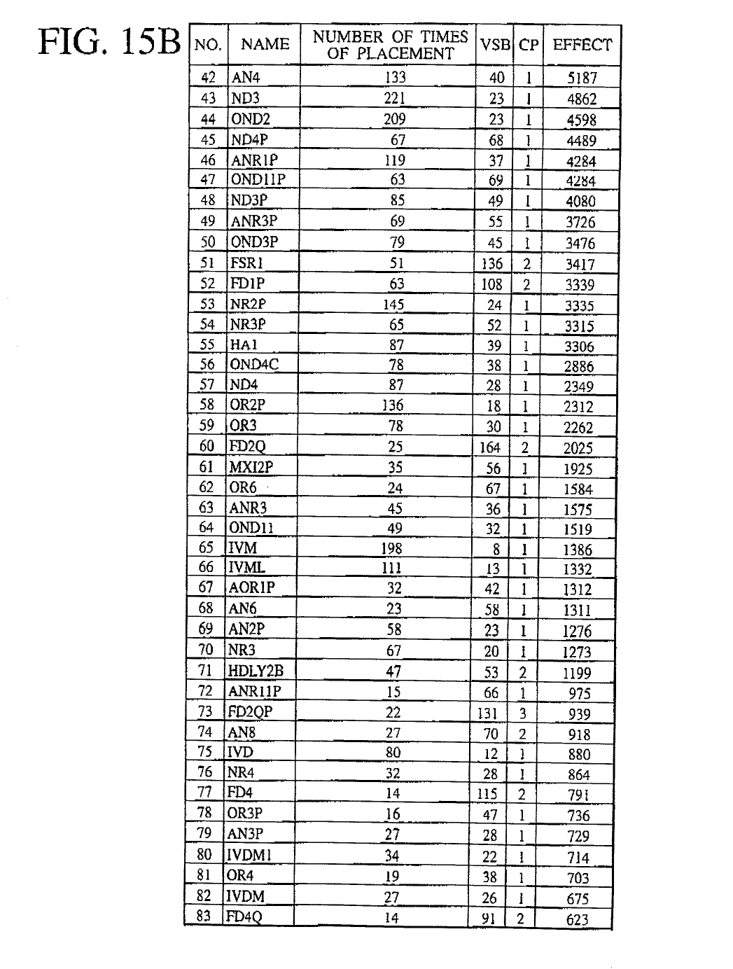

As for a portion designed by automatic P&R of a certain functional block of a certain logic product among these products, actual design data was examined. The lists of standard cells used in this functional block are shown in FIGS. 15A and 15B. Although 101 types of standard cells of No. 1 to No. 101 are actually included, FIGS. 15A and 15B show 83 types only. Here, the pattern of the gate layer of the cell named FD1Q of No. 1 is the D.F/F circuit shown in FIG. 45. Likewise, AN2 of No. 7 and IV of No. 28 are the AND circuit shown in FIG. 4A and the inverter shown in FIG. 4C, respectively.

All of the standard cells are set to have heights of 10 μm or less and to have different widths.

FIGS. 15A and 15B show the number of times of placing a cell (the frequency of using the cell), as well as the calculated number of shots for VSB exposure and the calculated number of shots for CP exposure when the magnitude of the largest electron beam irradiated on a sample is 10 μm square.

Since the height of each cell is 10 μm or less, the cell is subjected to CP exposure once. As for a cell having a width exceeding 10 μm, the cell is divided into two or more characters and placed on a CP aperture and a plurality of electron beam shots are needed to conduct CP exposure to the cell pattern. For example, since the cell FD1Q of No. 1 has a width of 13.2 μm, it is necessary to divide the cell into two characters and to place them on a CP aperture to conduct CP exposure.

A column of effect shows CP effectiveness in numeric values indicating an effect as to how many shots can be reduced by exposing the standard cell with CP exposure compared with a case of exposing the standard cell with VSB exposure. The CP effectiveness is calculated according to Expression 2 as follows:

(CP effectiveness)=(number of VSB shots−number of CP shots)×(frequency of placing cell)/(number of CP shots) (2)

In the list shown in FIGS. 15A and 15B, standard cells having higher CP effectiveness are allotted higher numbers in this order. The cell having the highest placement frequency is the inverter of No. 28. However, since the inverter is lower in the number of shots necessary for VSB exposure, the inverter is listed 28th.

There are 101 types of standard cells used in this function block. Considering the magnitudes of the respective cells, it was found that a total of 136 characters are required. In the list shown in FIGS. 15A and 15B, therefore, up to 83 standard cells requiring just 100 characters are shown.

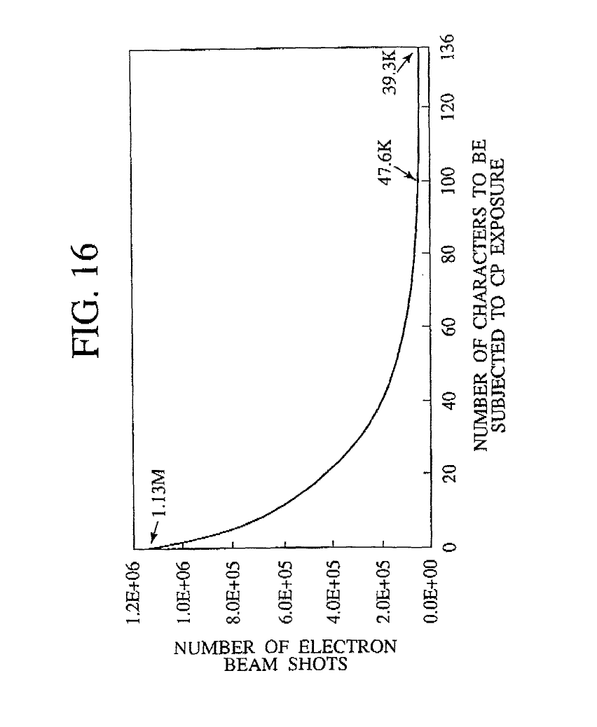

If the entire functional block is exposed only with VSB exposure, 1.13 M (mega) shots are required. If 136 characters, all of which can be subjected to CP exposure, are placed on a CP aperture, it suffices to irradiate 39.3 K (kilo) shots. In other words, if CP exposure is only conducted to expose all the standard cells, the number of shots can be reduced to about 1/28 and the throughput of electron beam exposure can be thereby greatly improved. Further, if all of the standard cells are not subjected to CP exposure, 83 standard cells which provide 100 characters are subjected to CP exposure and the remaining standard cells are subjected to VSB exposure, then the number of shots amounts to 47.6 K. Even so, the number of shots can be reduced to about 1/23 compared with a case where only VSB exposure is conducted, and the throughput of electron beam exposure can be improved enough. This relationship is shown in FIG. 16 in which the horizontal axis indicates the number of characters subjected to CP exposure and the vertical axis indicates the number of electron beam shots necessary to expose the overall functional block.

As can be seen, each of the standard cells included in the standard cell libraries 1 and 2 used to design logic products such as an ASIC and system LSI is made into a character subjected to CP exposure, whereby it is possible to manufacture a CP aperture 44 or a CP aperture block 3, as shown in FIG. 12 or 13, corresponding to the standard cells subjected to CP exposure.

As for the functional block of a certain logic product, if 101 standard cells made into 136 characters which are necessary for exposure with electron beams of 10 μm square can be prepared on a CP aperture, the number of electron beam shots can be reduced to about 1/28 compared with a case of conducting only VSB exposure. If 83 types of standard cells which provide 100 characters are made into characters subjected to CP exposure, the number of electron beam shots can be reduced to about 1/23.

Standard cells allotted to characters subjected to CP exposure are basic circuit elements constituting a semiconductor device which cells do not have great change until the generation of logic products is changed and which are used in common among a plurality of logic products. For that reason, the standard cells are also referred to as primitive cells. A CP aperture 44 manufactured by placing these standard cells or primitive cells thereon can be used in common among a plurality of logic products. Due to this, the same CP aperture 44 can be always used for logic products designed using the same standard cell libraries 1 and 2, so that electron beam exposure can be promptly started when layout data 30 on a designed pattern can be generated in FIG. 9.

On the other hand, according to currently conducted light exposure, after layout data is generated, an optical proximity effect correction is made and then an exposure mask is manufactured. Due to this, it takes lots of time until exposure is actually started. By making the standard cells into characters subjected to CP exposure as stated above, the number of electron beam shots can be sufficiently reduced and it is, therefore, expected to improve throughput comparative to light exposure.

Further, even if the number of characters which can be placed or a CP aperture 44 is limited and all standard cells cannot be made into characters subjected to CP exposure, it is possible to check which standard cells within the standard cell libraries 1 and 2 are exposed with CP exposure so that the number of shots can be effectively reduced when exposure is conducted using the same CP aperture 44 for a plurality of products by checking the CP effectiveness of standard cells for a plurality of products.

At this moment, if placing the standard cells having high CP effectiveness to narrow the distances among them by, for example, placing the standard cells having higher CP effectiveness closer to the center of the aperture 44, the movement distance of an electron beam irradiated on the CP aperture 44 can be suppressed to be short and it is, therefore, possible to expect reduction in exposure time.

Moreover, by manufacturing a plurality of CP apertures 44 and replacing the CP apertures 44 when conducting electron beam exposure to a plurality of logic products so as to deal with a case where the tendency of standard cells to be used greatly differ among the plurality of logic products, it is possible to deal with more logic products. Furthermore, as shown in FIG. 12, if a plurality of CP aperture blocks 3 are prepared on the CP aperture 44 so as to deal with a plurality of character selection deflected regions, an aperture block 3 to be used may be selected from among the plural blocks whenever each logic product is subjected to exposure.

In the libraries 1 and 2, the shapes of patterns are defined in the respective standard cells. Characters subjected to CP exposure are set as the units of the respective standard cells. Since the pattern shape 6 is defined on the CP aperture block 3 as shown in FIG. 13 for each standard cell subjected to CP exposure, it suffices to know only the positions at which the respective cells are placed on the chip in the pattern data. For example, even for pattern data of normally used GDSII STREAM type, it suffices to know only the positions at which standard cells subjected to CP exposure are placed, As for a pattern for VSB exposure, by contrast, it is required to form the pattern shape into a vertex coordinate string type or the like.

Moreover, it is preferable to prepare electron beam exposure standard cell libraries 1 and 2, and to conduct logic synthesis in the step S3, automatic P&R in the step S5 and the generation of layout data 30 in the step S6 shown in FIG. 9 so as to make a designed simpler, simplify the conversion of the layout pattern data 30 thus generated into exposure data in each electron beam exposure and to thereby reduce time required for conversion.

The electron beam exposure standard cell libraries 1 and 2 may be constituted to indicate which of the standard cells in the libraries 1 and 2 are made into characters subjected to CP exposure and the positions of these standard cells on the CP aperture 44. That is, as shown in FIG. 11, the shape of each standard cell subjected to CP exposure is defined on the aperture 44, pattern shape information is, therefore, unnecessary and each cell has three items of information as follows:

1. The designation of the position of the cell at which the cell is placed on the CP aperture 44.

2. Parameters, such as parameters of delay, resistance, capacity and the like, necessary to conduct timing analysis and logic simulation for an electron circuit.

3. The input and output positions of signals which are referred to when conducting automatic placement and routing (P&R) or particularly when wirings are provided.

If a plurality of CP apertures 44 are provided or a plurality of aperture blocks 3 are provided on a CP aperture 4, the designation of the CP aperture 44 or aperture block 3 including the shape of the cell is also included in the designation of the placement position in the above item 1 information. Accordingly, in case of normal standard cell libraries 1 and 2, each cell has information on the position of the cell on the CP aperture in the item 1 above instead of the pattern shape of the cell. However, if a plurality of CP apertures 44 are provided, exposure is conducted with either CP exposure or VSB exposure depending on the CP aperture 44 to be used. Due to this, it is necessary to include information on a pattern shape as usual. As for a pattern which is not provided on the CP aperture 44, the cell libraries 1 and 2 are the same as normal standard cell libraries. That is, it can be said that besides normal standard cells, information on the positions of cells subjected to CP exposure on a CP aperture is added to the electron beam exposure standard cell libraries 1 and 2.

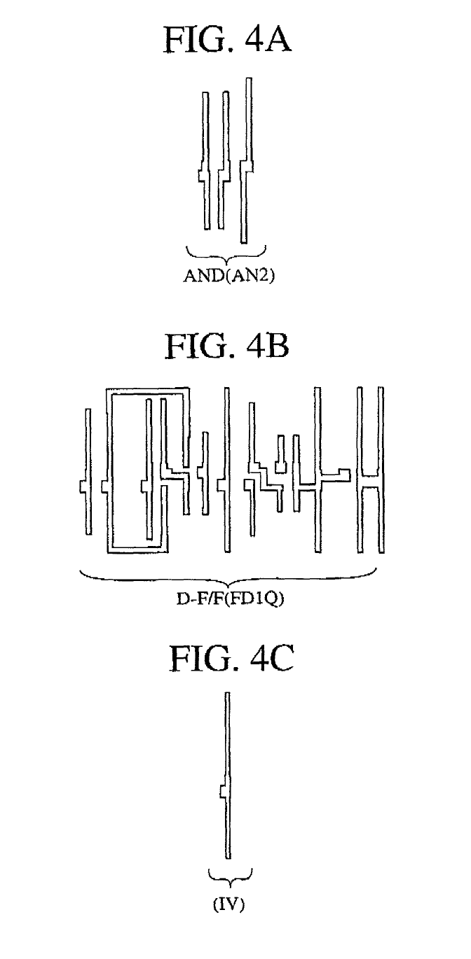

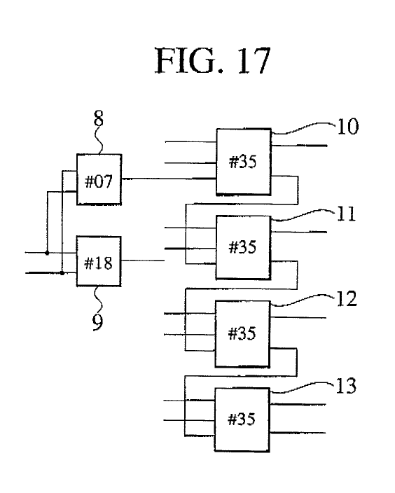

If the electron beam exposure standard cell libraries 1 and 2 as described are used, the net list generated by the logic synthesis in the step S3 of FIG. 9 shows in FIG. 17. The net list shown in FIG. 17 shows the manner in which the input and output terminals of the respective cells are connected to the other cells as in the case of the conventional net list shown in FIG. 3. In the conventional net list shown in FIG. 3, the respective cells are described by their names. In the net list according to this embodiment as shown in FIG. 17, by contrast, the cells 8 to 13 subjected to CP exposure are described by their positions on the aperture (or aperture block) or by the identification numbers (corresponding to the numbers (NO.) shown in FIGS. 15A and 15B) of characters subjected to CP exposure.

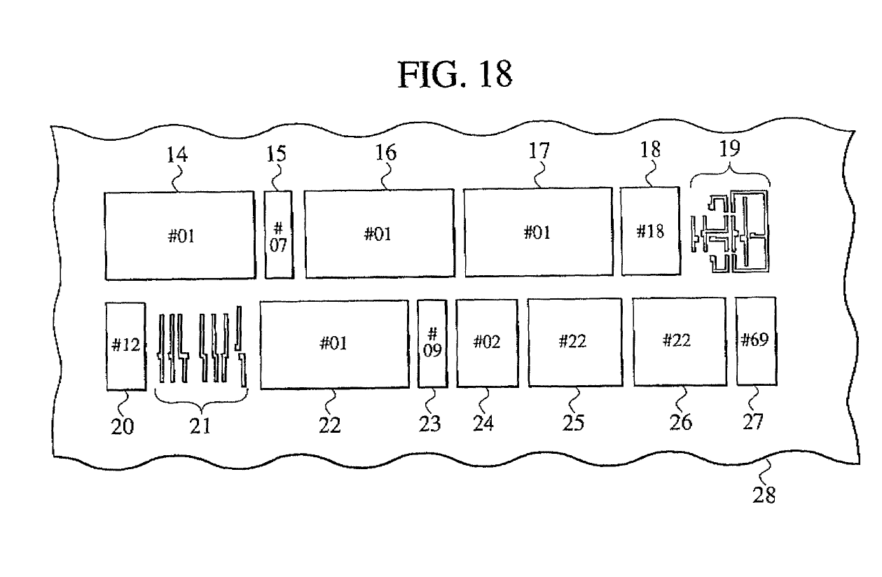

Moreover, the layout data 30 generated in the step S6 after the placement and routing (P&R) in the step S5 of FIG. 9 is pattern data 28 which can replace cells 14 to 18, 20 and 22 to 27 subjected to CP exposure by their positions on the aperture 44 (or aperture block 3) or by character numbers #01, #07, #18, #12, #09, #02, #22, #69 as shown in FIG. 18. Compared with the conventional layout data 65 shown in FIG. 5A, information on graphics in the cells on the cells 14 to 18. 20 and 22 to 27 can be omitted. It is noted that the cells 19 and 21 are subjected to VSB exposure.

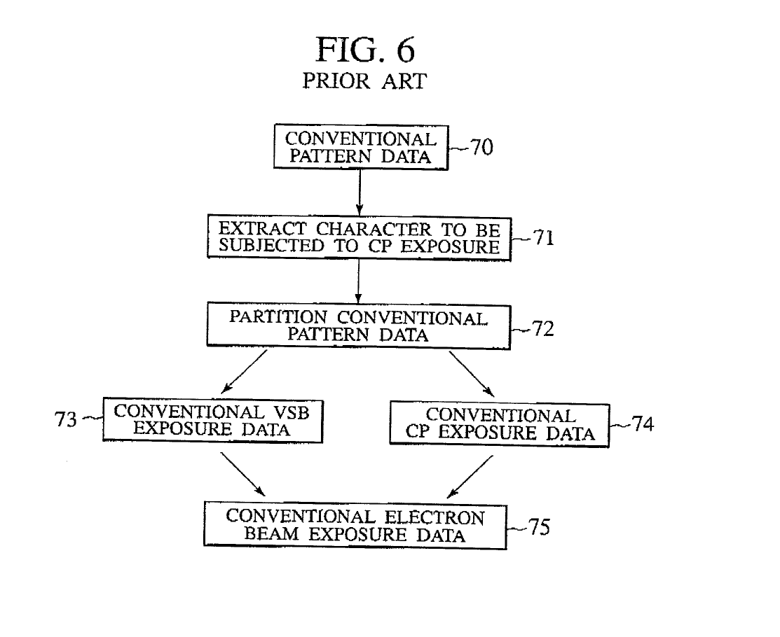

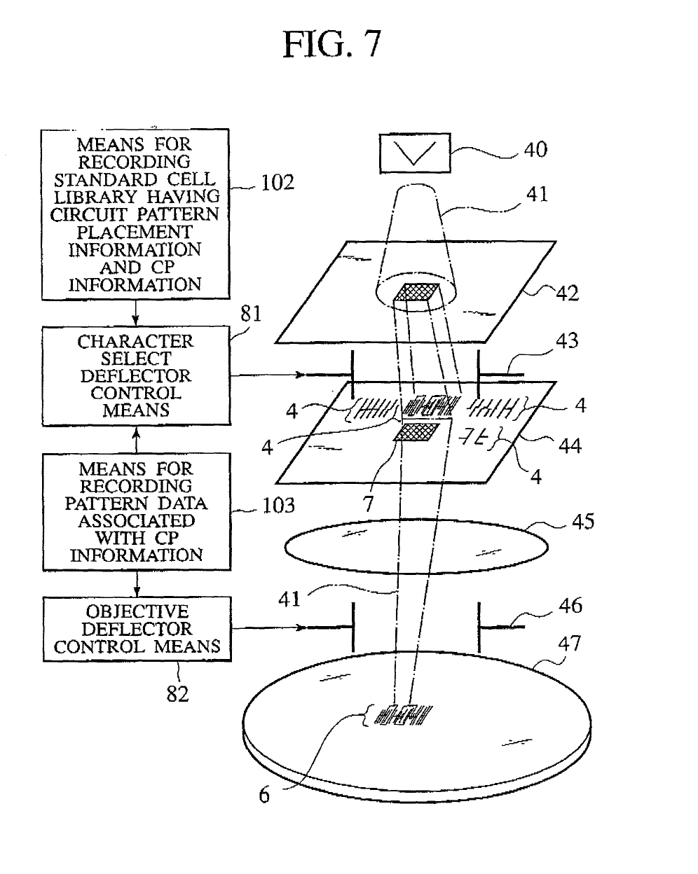

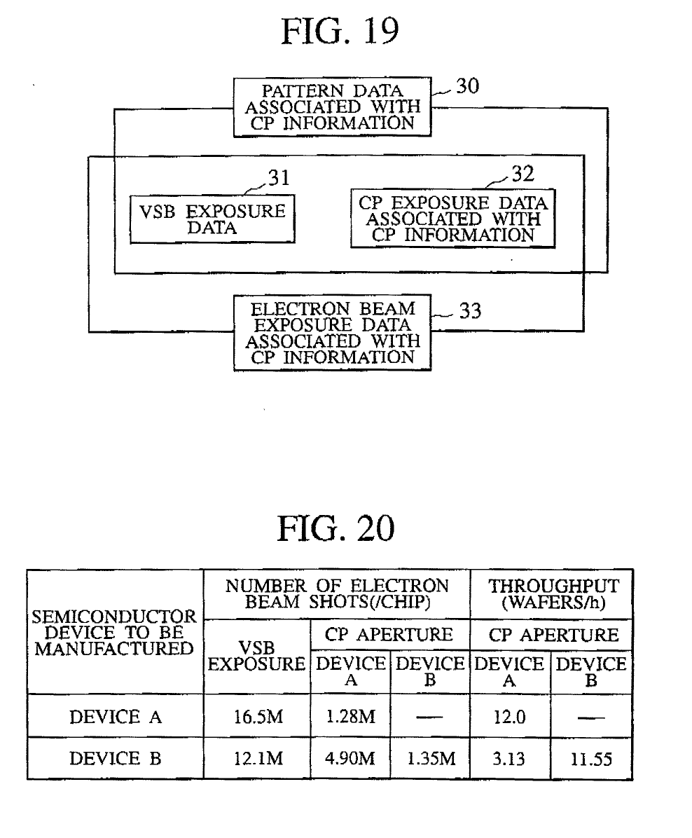

The layout pattern data 70 is conventionally converted into exposure data 75 for the electron beam exposure according to the flow shown in FIG. 6. According to the present invention, by contrast, it is known in advance which patterns are exposed with CP exposure. Polygon data, such as a vertex string forming each pattern, in the actual pattern data 30 is provided only for patterns for VSB exposure. Due to this, it is considered that the pattern data 30 associated with CP information is already divided into CP exposure data 32 associated with CP information and VSB exposure data 31 as shown in FIG. 19. Thus, it is possible to dispense with the step of extracting the CP exposure characters as denoted by reference symbol 71 and the step of dividing pattern data into VSB exposure data 73 and CP exposure data 74 as denoted by reference symbol 72 in FIG. 6. Thus, load on a computer conducting data conversion is reduced and time required for data conversion is shortened. It is noted that the data conversion is executed by the conversion means 92 constituting the exposure pattern data generation apparatus 83 and converting portions other than the standard cells subjected to CP exposure into VSB exposure data. That is to say, the VSB exposure data 31 is converted by the conversion means 92, whereby the converted data 31 and the CP exposure data 32 can constitute electron beam exposure data 33 associated with CP information.

As can be seen from the above, it suffices that the electron beam exposure standard cell libraries 1 and 2 have at least the following four items of information:

1. Names of standard cells.

2. Positional information as to at which positions of which CP aperture 44, shaping holes 4 forming the electron beam 41 into the standard cell shapes are formed. A plurality of positional information can be provided in the form of the positions of the cells on each CP aperture 44 for a plurality of CP apertures 44. In addition, as for a standard cell which has a low effect of reducing the number of shots even by conducting CP exposure, information on the position at which the standard cell is placed on the CP aperture 33 is not provided in the libraries because only VSB exposure is conducted.

3. Parameters necessary for timing analysis and logic simulation.

4. The positions of inputs and output used when conducting automatic placement.

These libraries 1 and 2 can be easily generated from the conventional libraries only by adding aperture information in the item 2 above.

In layout pattern data 30 on a logic product manufactured by conducting logic synthesis and automatic P&R using the electron beam exposure standard cell libraries 1 and 2, standard cells subjected to CP exposure are defined in advance. It is, therefore, unnecessary to extract characters subjected to CP exposure from the pattern data 30 and it is possible to reduce the number of data conversion steps compared with the conventional steps. Besides, since it is presumed that the existing generalized CP aperture 44 is used, it is unnecessary to manufacture a CP aperture 44 for each product, thereby making it possible to not only reduce mask production cost but also start electron beam exposure right after the generation of the pattern data 30 and therefore reduce time required since a product is ordered until the product is completed.

Furthermore, by using these electron beam exposure standard cell libraries 1 and 2 and the generalized CP aperture 44, it is possible to eliminate polygon data on the standard cells subjected to CP exposure so as to prevent the data from becoming very large in size. In case of current pattern data of GDSII STREAM type, if all layers are included, the size of the data sometimes becomes in the order of several hundreds of megabytes to gigabytes. According to this method, by contrast, in case of the pattern data 30 which file size has been reduced, it is possible to download or upload design data using a network such as the Internet in a short period of time, whereby it is possible to order a product outside a company and to execute process outside the company relatively easily, which operations have been conventionally difficult.

In a CP type charged beam exposure method for shaping a charged beam into a desired shape using an aperture and conducting demagnifying irradiation and exposure to a sample, a desired shape is formed into standard cell used for device design using a shaping hole 4 for shaping a charged beam. By doing so, logic product layout pattern data according to this invention is defined for standard cells subjected to CP exposure in advance. Thus, it is unnecessary to extract characters subjected for CP exposure from the pattern data. Besides, since it is presumed to use an existing, generalized CP aperture, it is unnecessary to manufacture a CP aperture for each product. This can reduce mask production cost. In addition, since it is possible to start electron beam exposure right after generating the pattern data, time required since a product is ordered until the product is completed can be shortened.

In a series of exposure steps of manufacturing a semiconductor device, the semiconductor device or photomask can be manufactured by executing a step of conducting logic synthesis for the semiconductor device using information on the standard cells of the semiconductor device, a step of placing and routing the standard cells using information on the standard cells and a step of placing and routing the standard cells by shaping a charged beam into standard cell shapes and conducting demagnifying irradiation and exposure to a semiconductor substrate or to a photomask substrate. Thus, it is possible to adopt CP exposure in the series of exposure steps of manufacturing the semiconductor device or photomask and to promptly manufacture the photomask to be used.

### Example 2 of Generating Exposure Pattern Data

Next, a method of generating pattern data 30 suited for CP type electron beam exposure will be described. The generation method is conducted according to a flow chart shown in FIG. 14. By way of example, description will be given to a case of generating pattern data 30 on a certain semiconductor device B while a CP aperture 44 is prepared.

As shown in FIG. 20, if there exists a CP aperture 44 manufactured for use in a semiconductor device A, pattern data 30 on another semiconductor device B is generated using this CP aperture 44. The allowed exposure throughput of the device B is a minimum of 10 wafers/h. While presuming process and an electron beam exposure to be used, the number of shots of the device A was calculated and converted into throughput of 12 wafers/h.

First, in the step sit of FIG. 14, logic synthesis is conducted In the form of a logic expression described in the logic design shown in FIG. 9 using the CP aperture which was manufactured for the device A. At this moment, the area of an electronic circuit to be synthesized, the operating frequency of the circuit and the like are designated as constraints.

In the step S12, since the number of CP apertures 44 manufactured for the device A is only one, the step S13 follows.

In the step S13, CP apertures which satisfy the designated constraints are extracted from a synthesized net list.

In the step S14, it is judged whether or not there is a CP apertures 44 satisfying the constraints if logic synthesis is conducted using only the cells placed on the CP aperture 44. Namely, when designing the device B, logic synthesis is conducted by assuming that the CP aperture 44 to be used was manufactured for the device A. However, it was impossible to generate a net list using only the CP aperture 44 manufactured for the device A. Since the CP aperture 44 manufactured for the device A does not satisfy the constraints, the step S17 follows.

In the step S17, the constraint of using only the cells on the CP aperture 44 manufactured for the device A is eliminated and logic synthesis is conducted again.

In the step S18, the number of electron beam shots in case of conducting exposure using the existing CP aperture 44 manufactured for the device A Is calculated for the patterns synthesized in the step S17. At this moment, the number of electron beam shots is calculated for the cells placed on the CP aperture 44 to be used by assuming that CP exposure is conducted and for the other cells and by assuming that VSB exposure is conducted. That is to say, the number of shots is calculated by assuming that among the standard cells used in the circuit, the cells placed on the CP aperture 44 manufactured for the device A are subjected to CP exposure and that the other cells are subjected to VSB exposure. As a result, it is found that the number of shots is 4.90 M shots per chip.

In the step S19, the calculation of the number of shots in the step S18 is conducted to the CP aperture 44 manufactured for the existing device A.

In the step S20, since there is only one CP aperture, the CP aperture 44 manufactured for the device A is selected.

In the step S21, if the number of shots for the CP aperture selected in the step S20 is converted into throughput according to Expression 1, the throughput is 3.13 wafers/h.

In the step S22, the throughput calculated in the step S21 is lower than desired throughput. Therefore, the step S23 follows.

In the step S23, a CP aperture 44 for the device B is manufactured. As shown in FIGS. 15A and 15B, the standard cells having higher CP effectiveness to those having lower CP effectiveness included in the net list synthesized in the step S17 are extracted in this order and a new CP aperture 44 is manufactured.

In the step S24, using the new CP aperture 44, pattern data 30 is generated by conducting P&R. The number of shots in this case is calculated as 1.35 M shots/chip and throughput is 11.55/h. Then, the processing flow of FIG. 14 is finished.

By following this flow, when the devices B is designed, it is possible to easily judge which CP aperture 44 is used to conduct electron beam exposure or whether a new CP aperture 44 should be manufactured or not. In this example, it is judged that it is necessary to manufacture a new aperture 44.

As stated so far, according to the present invention, it can provide a charged beam exposure capable of conducting CP exposure even for an integrated circuit, such as a logic product, which has a few repetitive patterns, capable of obtaining the effect of improving throughput by conducting the CP exposure and capable of easily generating charged beam exposure data.

According to the present invention, it can provide a exposure pattern data generation method capable of conducting CP exposure even for an integrated circuit, such as a logic product, which has a few repetitive patterns, capable of obtaining the effect of improving throughput by conducting the CP exposure and capable of easily generating charged beam exposure data.

Various modifications will become possible for those skilled in the art after receiving the teachings of the present disclosure without departing from the scope thereof.

What is claimed is:

1. A charged beam exposure comprising:
a beam generation source generating a charged beam;
a Character Projection (CP) aperture having shaping holes of the charged beam having shapes of standard cells used for logic design of a system;
standard cell library recording means for recording first placement positions of the shaping holes on said CP aperture;
pattern data recording means for recording second placement positions of the standard cells on a substrate, the second placement positions associated with the first placement positions;
a character select deflector irradiating the charged beam onto the shaping holes at the first placement positions; and
an objective deflector irradiating the charged beam onto the second placement positions on the substrate.

2. An exposure as in claim 1, further comprising:
a first shaping aperture rectangularly shaping an irradiation pattern of the charged beam to said CP aperture.

3. An exposure as in claim 1, further comprising:
a demagnifying lens demagnifying the irradiation pattern of the electron beam on the substrate.

4. An exposure as in claim 1, wherein
said standard cell library recording means further records input and output positions of signals of the standard cells.

5. An exposure as in claim 1, wherein
said CP aperture further has an opening for a variable shaped beam (VSB).

6. An exposure as in claim 1, wherein
the shaping holes have a shape of the standard cell having a higher frequency of use or a shape of the standard cell having a higher effect of reducing the number of shots by CP exposure than by VSB exposure.

7. An exposure pattern data generation apparatus comprising:
Character Projection (CP) aperture decision means for conducting logic synthesis for CP apertures using standard cells corresponding to shaping holes placed on first placement positions on the respective CP apertures, and for selecting the CP aperture used for exposure; and
placement and routing means for calculating second placement positions of the standard cells on a substrate, the standard cells Corresponding to the shaping holes provided on the selected CP aperture.

8. An apparatus as in claim 7, further comprising:
VSB exposure data conversion means for converting data into data capable of being used by an exposure to conduct variable shaped beam (VSB) exposure to the standard cells which cannot be subjected to exposure using the shaping holes.

9. An apparatus as in claim 7, wherein
said CP aperture decision means comprises:
standard cell extraction means for extracting the standard cells;
logic synthesis means for conducting synthesized logic using the extracted standard cells; and
constraints and the like determination means for determining whether the logic synthesis satisfies a specification.

10. An apparatus as in claim 9, wherein
said CP aperture decision means further comprises:
CP aperture creation means for creating a new CP aperture if the CP apertures cannot satisfy the specification.

11. An apparatus as in claim 7, wherein
said placement and routing means calculates wiring routes among the placed standard cells.

12. An apparatus as in claim 7, further comprising:
first standard cell library recording means for recording magnitudes, functions and performances of the standard cells, an identification code of the CP aperture on which the shaping holes having the shapes of the standard cells are formed and the first placement positions, and for providing the recorded magnitudes, functions and performances of the standard cells, the identification code and the first placement positions to said CP aperture decision means.

13. An apparatus as in claim 7, further comprising:
second standard cell library recording means for recording shapes and magnitudes of outlines of the standard cells, positions of input and output signals, an Identification code of the CP aperture on which the shaping holes having the shapes of the standard cells are formed and the first placement positions, and for providing the recorded shapes and magnitudes of the outlines of the standard cells, positions of the input and output signals, identification code and the first placement positions to said placement and routing means.

14. An apparatus as in claim 13, further comprising:
pattern data recording means for recording the second is placement positions, the identification code and wiring routes among the standard cells provided from said placement and routing means.

15. An exposure pattern data generation method comprising:
conducting logic synthesis for Character Projection (CP) apertures using standard cells corresponding to shaping holes placed at first placement positions on the respective CP apertures;
selecting a CP aperture used for exposure from the CP apertures; and
calculating second placement positions of the standard cells on a substrate, the standard cells corresponding to the shaping holes provided on the selected CP aperture.

16. A method as in claim 15, further comprising:
converting data into data capable of being used by an exposure to conduct variable shaped beam (VSB) exposure to the standard cells which cannot be subjected to exposure using the shaping holes.

17. A method as in claim 15, wherein
said conducting logic synthesis for CP apertures using standard cells corresponding to shaping holes placed at first placement positions on the respective CP apertures comprises:
extracting the standard cells; and
conducting logic synthesis using the extracted standard cells, and
said selecting a CP aperture used for exposure from the CP apertures comprises:
determining whether the synthesized logic satisfies a specification.

18. A method as in claim 15, wherein
said conducting logic synthesis for CP apertures using standard cells corresponding to shaping holes placed at first placement positions on the respective CP apertures further comprises:
creating a new CP aperture if the CP apertures cannot satisfy the specification.

19. A method as in claim 15, wherein
the calculating second placement positions of the standard cells on a substrate, the standard cells corresponding to the shaping holes provided on the selected CP aperture further comprises:
calculating wiring routes among the placed standard cells.

20. A method as in claim 15, further comprising:
recording magnitudes, functions, outline shapes and outline magnitudes of the standard cells, positions of input and output signals, identification codes of the CP apertures on which the shaping holes having the shapes of the standard cells are formed and the first placement positions.

21. A method as in claim 15, further comprising:
recording the second placement positions, identification codes and wiring routs among the standard cells.
